(19) United States
(12) Plant Patent Application Publication
(10) Pub. No.: US 2020/0154623 P1
(43) Pub. Date: May 14, 2020

(54) Cannabis plant named 'MR2018003'

(50) ;

(71) Applicant: MedReleaf Corp., Toronto (CA)

(72) Inventors: Jeremy N. Friedberg, Guelph (CA); Andrew Hand, Bradford (CA); Alan Graziano, Toronto (CA)

(73) Assignee: MedReleaf Corp., Toronto (CA)

(21) Appl. No.: 16/602,633

(22) Filed: Nov. 13, 2019

(30) Foreign Application Priority Data
Nov. 14, 2018 (CA) 18-9644

(51) Int. Cl. A01H 6/28

(52) U.S. Cl. PLT/263.1

(57) ABSTRACT

A new cultivar of Cannabis named ‘MR2019003’ that is characterized by 16% THC and 0% CBD by dry weight, resistance to microbial growth, high yield and low intra-flower leaves.

Genus and species: Cannabis sativa.

Variety denomination: ‘MR2018003’.

### CROSS-REFERENCE TO RELATED APPLICATIONS

This application claims the benefit of priority under 35 US.S.C 199(f) to Canadian Plant Breeders' Rights Application Number 18-9644, which was filed for the instant plant variety on Nov. 14, 2018.

### BACKGROUND OF THE INVENTION

The present invention relates to a new and distinct Cannabis (Cannabis sativa) cultivar designated as ‘MR2018003’.

‘MR2018003’ is a selection resulting from a controlled-cross made in in Markham, Ontario, Canada, between the female Cannabis sativa variety ‘Pakistan Chitral Kush Landrace’ and the male Cannabis sativa variety ‘UK Cheese’.

Seeds from the cross were sown in Markham, Ontario, Canada and plants were screened for a number of traits including tetrahydrocannabinol (THC) and cannabidiol (CBD) levels, yield, flowering time, disease resistance and flower morphology. An individual plant having about 16% THC and 0% CBD by dry weight and have significant resistance to microbial growth was chosen to be ‘MR2018003’.

In May 2016, ‘MR2018003’ was first asexually propagated by apical stem cuttings approximately 10 cm long and having multiple auxiliary meristems, in Markham, Ontario, Canada. ‘MR2018003’ is stable and reproduces true to type in successive generations of asexual reproduction.

### BRIEF SUMMARY OF THE INVENTION

This invention relates to a new and distinctive Cannabis cultivar designated as ‘MR2018003’.

‘MR2018003’ exhibits approximately 16% THC and 0% CBD by dry weight. ‘MR2018003’ also exhibits resistance to microbial growth, high yield and low intra-flower leaves.

### BRIEF DESCRIPTION OF THE DRAWINGS

The accompanying color photographs depict characteristics of ‘MR2018003’ as nearly true as possible to make color reproductions. The photographs are of plants aged 8 to 9 weeks post-floral transition and grown in flower rooms under standard cultivation methods in Bradford, Ontario Canada.

FIG. 1 shows multiple, whole plant clones of ‘MR2018003’.

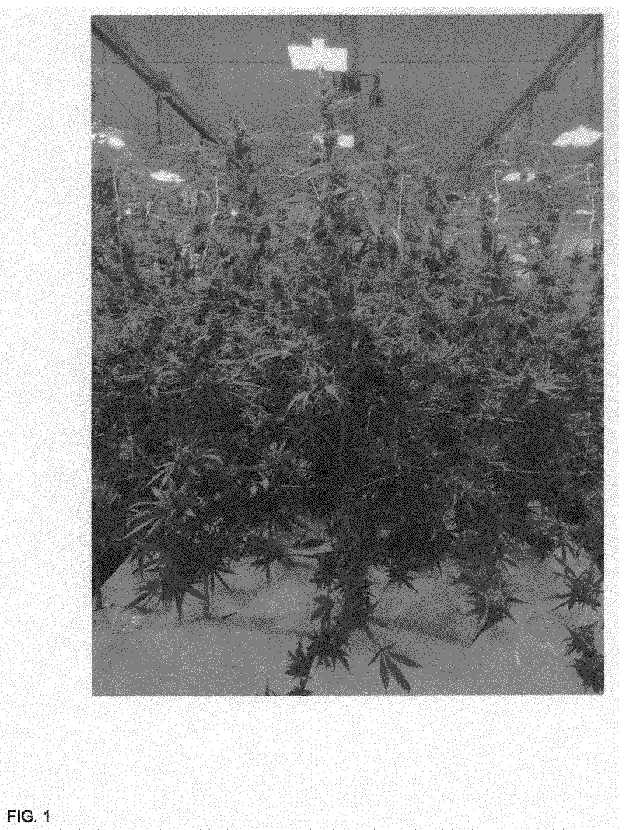

FIG. 2 shows a close-up view of a single inflorescence and foliage of ‘MR2018003’.

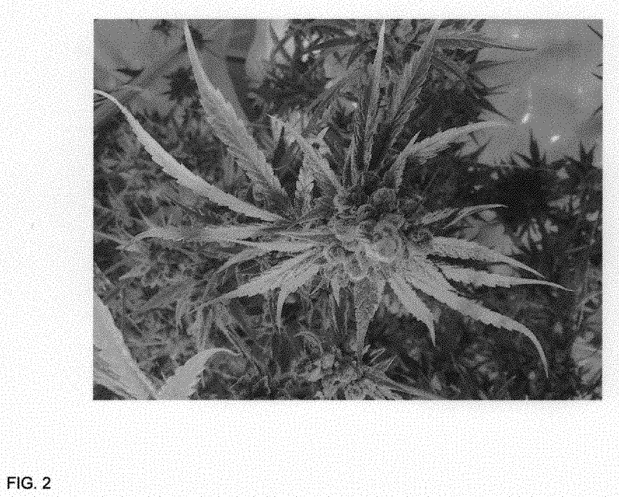

FIG. 3 shows a close-up view of foliage of ‘MR2018003’.

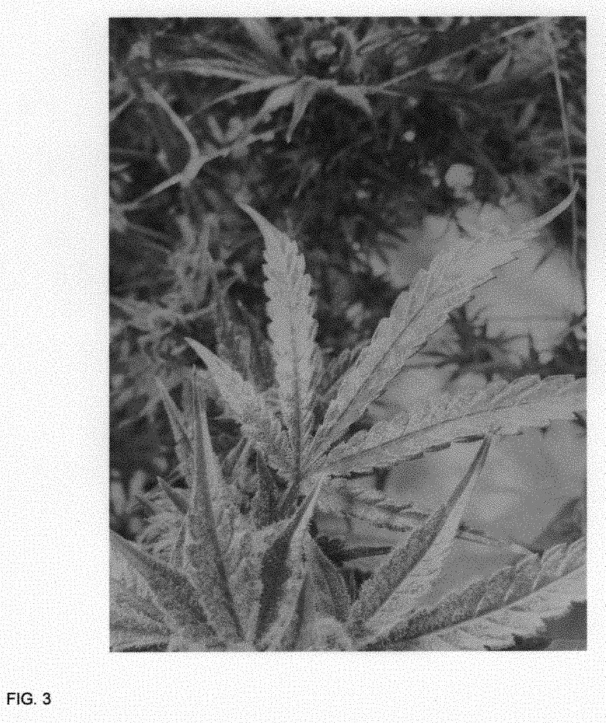

### DETAILED BOTANICAL DESCRIPTION

The following is a detailed description of the new cultivar ‘MR2018003’.

‘MR2018003’ has not been tested under all possible environmental conditions. Phenotypic differences may be observed with variations in environment without any variance in genotype.

The traits of ‘MR2018003’ have been repeatedly observed and represent the distinguishing characteristics of ‘MR2018003’.

The data that follows was collected in Markham, Ontario Canada and Bradford, Ontario, Canada from plants aged 8 to 9 weeks post-floral transition.

Plants were flowered under standard indoor environmental conditions with High Pressure Sodium lamps. Clones were cut from healthy mothers and allowed to roots for 14 to 18 days prior to being introduced to the flower room. The room was maintained at 18 hour days/6 hour nights for a week to allow the cuttings to establish adequate vegetative tissue. The floral transition was initiated by switching to 12 hour days and 12 hour nights. Flowering lasted 9 weeks before harvest, at which point morphological measurements were taken, samples were taken for chemical analysis, and yield was quantified.

Standard in-house developed nutrients were used throughout growth, and the plants were defoliated and pruned as necessary throughout the cycle.

In the following description, the color determination is in accordance with the 2017 Munsell Plant Tissue Color Book, except where general color terms of ordinary dictionary significance are used.
Classification:
   Denomination.—‘MR2018003’.
   Family.—Cannabacaeae.
   Genus.—Cannabis.
   Species.—Cannabis sativa.
   Common Name.—Marijuana.
Parentage: Female Cannabis sativa variety ‘Pakistan Chitral Kush Landrace’ and male Cannabis sativa variety ‘UK Cheese’.
Propagation: ‘MR2018003’ is asexually (clonally) propagated from vegetative cuttings. It takes approximately 18 days to produce a rooted young plant. Roots are fine and well-branched.
Plant:
   Height.—78 cm-120 cm.
   Width.—40 cm-55 cm.
Stems:
   Length/height.—78 cm-110 cm.
   Width/diameter.—4 mm-9 mm.
   Color.—2.5 GY 5/6, 5GY 7/8, 5RP 3/4.
   Shape.—Tubular.
   Texture.—Fibrous and Pubescent.
   Lateral branch length.—15 cm-48 cm.
   Average number of nodes.—29.2.
   Average intermodal space.—6.2 cm.
Foliage:
   Type/form.—Palmately Compound.
   Arrangement.—Alternate.
   Attachment.—Petiolate.
   Leaf width.—16 cm-18 cm for fully mature leaves.
   Leaf length.—13 cm-16 cm for fully mature leaves.
   Number of leaflets per leaf.—5-7.
   Leaflet shape.—Lanceolate.
   Leaflet length.—13 cm-14 cm for longer leaflets, and 3 cm-5 cm for shorter leaflets.
   Leaflet width.—1.5 cm-2.0 cm for larger leaflets, and 0.3 cm-1.0 cm for smaller leaflets.
   Leaflet margin.—Serrate.
   Leaflet apex.—Acuminate.
   Leaflet base.—Attenuate.
   Leaflet color, upper surface.—5GY 4/4, 7.5 GY 3/4.
   Leaflet color, lower surface.—7.5 GY 6/8.
   Venation pattern and description, upper and lower leaflet surfaces.—Pinnate.
   Texture (both surfaces).—Pubescent, primarily on the abaxial side and along leaf vasculature.
   Fragrance.—Sweet, berry, fruity, cheesy.
   Stipules.—Present, 2 per node on either side of a petiole, attenuated.
Petiole:
   Length.—2 cm-6 cm.
   Diameter.—1.6 mm-2.5 mm.
   Texture.—Fine, short non-glandular trichomes.
   Color.—5RP 4/6.
Inflorescence:
   Blooming habit.—Short day photo period sensitive.
   Blooming period.—50 days.
   Attachment.—Subsessile.
   Bracts.—Covered with trichomes and resin glands with 2-3 stigmas emerging in each.
   Fragrance.—Sweet, berry, and fruity.
   Inflorescence color.—7.5 GY 5/2.
   Number of inflorescences per plant.—65-95.
   Diameter.—3 cm-4.5 cm.
   Length.—4 cm-7 cm.
   Inflorescence anthocyanin.—Medium to strong anthocyanin presence. 5 RP 3/2.
Reproductive organs:
   Pistils.—1.
   Quantity (of stigmas) per flower.—2-3.
   Stigma color.—5 YR 5/10 at maturity/senescence; 7.5 GY 7/4 for younger plants.
Disease and insect/pest resistance: ‘MR2018003’ exhibits resistance to aerobic bacteria, yeast, mold and coliform bacteria as shown in Table 1.

TABLE 1
‘MR2018003’ resistance to
aerobic bacteria, yeast,
mold and coliform bacteria.

| | |
|---|---|
| Average Aerobic Bacteria Count | 145 |
| (per plant, in colony forming | |
| units (CFUs)) | |
| Average Yeast and Mold Count | 37.5 |
| (per plant, in CFUs) | |
| Average coliform bacteria count | 32.5 |
| (per plant, in CFUs) | |

### COMPARISON WITH PARENTAL LINES AND KNOWN VARIETY

‘MR2018003’ may be compared with its parental lines with respect to THC content. ‘MR2018003’ exhibits 16.4% THC by dry weight, the female parent exhibits 12% THC by dry weight and the male parent exhibits 16% THC by dry weight.

The closest variety of Cannabis known to the inventors is the commercial variety ‘Girl Scout Cookies’. ‘MR2018003’ can be distinguished from ‘Girl Scout Cookies’ by its increased resistance to microbial growth, as shown in Table 2.

TABLE 2
Comparison between ‘MR2018003’ and
‘Girl Scout Cookies’: Microbial resistance

| | | | Average | |
|---|---|---|---|---|
| | | Average | Yeast | Average |
| | # of | Aerobic | and | Coliform |
| | Cycles | Bacteria | Mole | Bacteria |
| Variety | Grown | Count* | Count** | Count* |
| MR2018003 | 5 | 145 | 37.5 | 32.5 |
| Girl Scout | 15 | 59270 | 52450 | 760 |
| Cookies | | | | |

**Aerobic Bacteria Count, Yeast and Mold Count and Coliform Bacteria Count were determined per plant, in colony forming units (CFUs).

Variety ‘MR2018003’ can also be distinguished from the variety ‘Girl Scout Cookies’ with respect to the morphological traits shown the Table 3.

TABLE 3
Comparison between ‘MR2018003’
and ‘Girl Scout Cookies’:
Morphological traits

| | | ‘Girl Scout |
|---|---|---|
| Characteristic | MR2018003 | Cookies’ |
| Overage | 87.9 cm | 69.6 cm |
| Average Height | | |
| Average Active | 71.9 cm | 56.5 cm |
| Height | | |
| Average # of | 29.2 | 22.3 |
| Nodes | | |
| Average | 6.2 cm | 6.6 cm |
| Internodal Space | | |
| Average Flower | 3.4 cm | 5.3 cm |
| Width | | |

‘MR2018003’ also has a higher yield and fewer intra-flower leaves than ‘Girl Scout Cookies’. The flowers of ‘MR2018003’ are dark purple in colour and have large, swollen calyxs. In contrast, the flowers of ‘Girl Scout Cookies’ are green in colour and the calyxs are not as large.

1. A new and distinct cultivar of Cannabis plant named ‘MR2018003’ as described and illustrated herein.
